(19) United States
(12) Patent Application Publication
(10) Pub. No.: US 2013/0330756 A1
(43) Pub. Date: Dec. 12, 2013

(54) DETECTION OF BACTERIA EXHIBITING ENZYMATIC RESISTANCE TO CARBAPENEMS

(71) Applicants: Sandrine Ghirardi, Saint Genis les Ollieres (FR); John Perry, Newcastle upon Tyne (GB); Gilles Zambardi, Chezeneuve (FR)

(72) Inventors: Sandrine Ghirardi, Saint Genis les Ollieres (FR); John Perry, Newcastle upon Tyne (GB); Gilles Zambardi, Chezeneuve (FR)

(73) Assignee: BIOMERIEUX, Marcy L'Etoile (FR)

(21) Appl. No.: 14/001,553

(22) PCT Filed: Mar. 16, 2012
(86) PCT No.: PCT/FR2012/050557

§ 371 Date: Aug. 26, 2013

(30) Foreign Application Priority Data
Mar. 25, 2011 (FR) 1152476

(51) Int. Cl. C12Q 1/04

(52) U.S. Cl. CPC C12Q 1/04
USPC 435/19; 435/18

(57) ABSTRACT

A process for detecting and/or identifying, in a biological sample, bacteria exhibiting a resistance to carbapenems by producing carbapenemase, includes: a) contacting said sample with a reaction medium including at least one carbapenem-class antibiotic, and cloxacillin; b) incubating the whole so as to allow the bacteria to grow; and c) detecting the strains exhibiting enzymatic resistance to carbapenems. Advantageously, the medium employed in step a) also contains phenylalanine-arginine-beta-naphthylamide (PAbetaN).

The present invention relates to a process for detecting and identifying bacteria which are resistant to carbapenems. More precisely, the process according to the invention aims to detect bacteria exhibiting enzymatic resistance to carbapenems.

The increase in the resistance to beta-lactam antibiotics, such as penicillins and cephalosporins, complicates the treatment of infections caused by strains of Gram-negative bacteria. These antibiotics are then replaced by other broad-spectrum antimicrobials. Amongst these broad-spectrum antimicrobials, carbapenems have taken an important role, especially for treating hospitalised patients. Carbapenems act against the majority of Gram-positive and Gram-negative aerobic bacteria, and on certain anaerobic bacteria.

However, more and more strains resistant to carbapenems are appearing in hospitalised patients.

The bacteria concerned are, non-exhaustively, Escherichia coli, Enterobacter cloacae, Enterobacter aerogenes, Citrobacter sp., Klebsiella pneumoniae, Klebsiella oxytoca, Pseudomonas aeruginosa, Providencia rettgeri, Pseudomonas putida, Stenotrophomonas maltophilia, Acinetobacter baumanii, Comamonas sp., Aeromonas sp., Morganella morganii, Enterococcus sp., Proteus mirabilis, Salmonella senftenberg, Serratia marcescens, Salmonella typhimurium, etc.

The reduced susceptibility to carbapenems can be due to:
the expression of a gene which is resistant to beta-lactams:
   (i) hyperproduction of ampC beta-lactamases and/or
   (ii) ESBL (extended-spectrum beta-lactamase),

combined with changes in the permeability of the cell wall (impermeability resistance) and/or with the active efflux of the antibiotics (Pages et al., 2009; PloS ONE, 4 (3)); and/or
the existence of enzymes which break down carbapenems, called carbapenemases.

The carbapenemase genes may be present in chromosomes and/or in plasmids. Due to this presence in the form of plasmids, these kinds of enzymatic resistances are capable of spreading to a great extent, and consequently pose a major risk in epidemiological terms. Since membrane-inpermeability resistance to carbapenems cannot spread, it is recommended as part of diagnosis, monitoring carriage or hygiene, to be able to distinguish between the two types of resistance.

When faced with strains of bacteria which are carbapenem-resistant, the person skilled in the art has difficulty in easily differentiating strains of carbapenemase-producing bacteria from strains which are impermeability-resistant. Since the two types of bacteria are capable of developing in the presence of a carbapenem, the only way of differentiating them at present consists in performing additional tests. Amongst these additional tests, it is possible to mention: the modified Hodge test (CLSI M100-S20: Performance Standards for Antimicrobial Susceptibility Testing; Twentieth Informational Supplement. January 2010. Supplemental Table 2A-S2), agar diffusion (or combined discs) synergy tests, employing inhibitors combined with carbapenem, for example EDTA for class-B carbapenemases, or phenyl-boronic acid for the detection of class-A carbapenemases. It is thus known to use discs combining meropenem and boronic acid compounds, as beta-lactamase inhibitors, for phenotype detection of KPC carbapenemases (Pournaras et al., 2010; J. Antimicrob. Chemotherapy, 65(7): 1319-1321). Similarly, Giske et al. tested the impact of cloxacillin on discriminating between KPC-producing strains and the strains combining loss of porins and hyperproduction of AmpC (Giske et al., 2010; Clin. Microbiol. Infect.; 17 (4): 552-6). In this case, discrimination is based on the inhibiting action of cloxacillin on the cephalosporinases coded for by the AmpC gene, which restores the action of beta-lactams. However, the result of these tests must often be confirmed by molecular biology techniques (for example PCR amplification targeting carbapenem-resistance genes).

A method of characterising by using a chromogenic medium comprising meropenem and/or ertapenem was suggested in Application WO 2010/010083 and implemented in the media CHROMagar® KPC (Samra et al., 2008; J. Clin. Microbiol., 46 (9): 3110-3111; CHROMagar™, Paris, France) and COLOREX™ KPC (BioMed Diagnostics Inc.). This medium does not permit the detection of all of the carbapenamase-producing strains, particularly metallo-beta-lactamases (Nordmann et al., 2011; J Clin Microbiol., 49(2): 718-721).

In the current epidemiological context, and particularly with the emergence of new resistances such as NDM-1, there is still a need to improve, in sensitivity and/or in specificity, the screening of all of the mechanisms of resistance linked to the production of the various types of carbapenemases.

The Applicant has shown that it is possible to improve the direct distinction, in other words particularly in a single screening device, between enzymatic resistance and the resistance by other mechanisms, impermeability amongst others, by inhibition of strains exhibiting impermeability resistance or another non-enzymatic resistance mechanism, without affecting the growth and the detection of the strains which are resistant through carbapenemase production. The Applicant has surprisingly observed an impact of cloxacillin, associated or not with PAbetaN, on carbapenem-resistant strains not producing carbapenemase and not producing AmpC.

In this respect, the present invention relates to a process for detecting and/or identifying, in a biological sample, bacteria exhibiting a resistance to carbapenems, comprising the steps consisting in:
a) contacting said sample with a reaction medium comprising at least one carbapenem-class antibiotic, and cloxacillin;
b) incubating the whole so as to allow the bacteria to grow;
c) detecting the strains exhibiting enzymatic resistance to carbapenems.

Advantageously, the medium employed in step a) also contains phenylalanine-arginine-beta-naphthylamide (PAbetaN).

The Applicant has surprisingly shown that the addition of cloxacillin or a combination of cloxacillin and PAbetaN into a chromogenic or non-chromogenic medium comprising carbapenems makes it possible to improve the sensitivity and the specificity of detection of bacteria which are resistant to carbapenems through carbapenemase production and to identify more specifically the carbapenemase-producing strains. More particularly, the method according to the invention makes it possible to differentiate among the carbapenem-resistant bacterial strains the carbapenemase-producing strains, strains exhibiting impermeability resistance or another non-enzymatic resistance mechanism. Thus the hygiene measures necessary for preventing the transmission of strains exhibiting enzymatic resistance can thus be implemented without delay.

According to a first embodiment, the present invention corresponds to a process for detecting and/or identifying, in a biological sample, bacteria exhibiting a resistance to carbapenems through carbapenemase production, comprising the steps consisting in:
a) contacting said sample with a reaction medium comprising at least one carbapenem-class antibiotic, and cloxacillin;
b) incubating the whole so as to allow the bacteria to grow;
c) detecting the strains exhibiting enzymatic resistance to carbapenems.

Advantageously, the medium employed in step a) also contains phenylalanine-arginine-beta-naphthylamide (PAbetaN).

According to a second embodiment, the present invention corresponds to a process for detecting and/or identifying, in a biological sample, bacteria exhibiting a resistance to carbapenems through carbapenemase production, comprising the steps consisting in:
a) contacting said sample with a reaction medium comprising at least one chromogenic substrate, at least one carbapenem-class antibiotic, and cloxacillin;
b) incubating the whole so as to allow the bacteria to grow;
c) detecting the strains exhibiting enzymatic resistance to carbapenems.

Advantageously, the medium employed in step a) also contains phenylalanine-arginine-beta-naphthylamide (PAbetaN).

Biological sample is to be understood to be a small part or small isolated quantity of an entity for analysis. This can be a clinical sample, human or animal, from a specimen of biological liquid, or a food sample, from any type of food, or a sample from the food production or processing environment. This sample can thus be liquid or solid. It is possible to cite in a non-limiting manner, a clinical sample of whole blood, serum, plasma, urines, faeces, specimens of nose, throat, skin, wounds, cerebrospinal fluid, a food sample of water, beverages such as milk, fruit juices, yoghurt, meat, eggs, vegetables, mayonnaise, cheese, fish, etc., a food sample from an animal feed, such as in particular an animal meal sample, or a sample for control of a surface area or water body. This specimen can be used such as it is or, prior to the analysis, undergo a preparation by enrichment, dilution, extraction, concentration or purification, in accordance with methods known to the person skilled in the art.

Reaction medium, is to be understood to be a medium comprising all the elements necessary for the expression of metabolism and/or for the growth of microorganisms. The reaction medium can be solid, semi-solid or liquid. Solid medium is understood to be a gelled medium, for example. Agar is the conventional gelling agent in microbiology for the culturing of microorganisms, but it is possible to use gelatine, agarose, or other natural or artificial gel-forming substances. A number of preparations are commercially available, for instance Columbia agar, Trypcase-soy agar, MacConkey agar, Mueller Hinton agar or more generally those described in the Handbook of Microbiological Media (CRC Press).

The reaction medium may comprise one or more elements in combination, such as amino acids, peptones, carbohydrates, nucleotides, minerals, vitamins, etc. The medium may also contain a dye. As an indication, possible dyes may be Evans blue, neutral red, sheep blood, horse blood, an opacifier such as titanium oxide, nitroaniline, malachite green, brilliant green, one or more metabolic indicators, one or more metabolic regulators, etc.

The reaction medium may be a revealing medium or a culturing and revealing medium. In the first case, the culturing of the microorganisms is performed before seeding and, in the second case, the detection and/or identification medium also constitutes the culture medium.

The person skilled in the art may also use a bi-plate, which makes it possible to easily compare two media, comprising different substrates or different selective mixtures, onto which the same biological sample will have been deposited.

The reaction medium may comprise one or more selective agents. Selective agent is to be understood to be any compound capable of preventing or slowing the growth of a microorganism other than the target microorganism. Without being limiting, a concentration of between 0.01 mg/l and 5 g/l is particularly suitable for the present invention.

As a selective agent, mention can be made of antibiotics, antifungals, bile salts, crystal violet, basic fuchsine, brilliant green, etc. Antibiotics are to be understood to be any compound capable of preventing or slowing the growth of a bacterium. In particular, they belong to the beta-lactams, glycopeptides, aminosides, polypeptides, sulfamides and quinolones groups. As an indication, it is in particular possible to mention the antibiotics cefotaxime, cefsulodin, ceftazidime, cefoxitin, ceftriaxone, cefpodoxime, aztreonam, vancomycin, gentamicin, Trimethoprim, tobramycin, moxalactam, fosfomycin, D-cycloserine, Polymyxin, Colistin, and quinolones such as nalidixic acid.

Antifungals are to be understood to be any compound capable of preventing or slowing the growth of a yeast or a mould. By way of indication, it is possible to mention in particular amphotericin B, fluconazole, itraconazole, voriconazole and cycloheximide.

The carbapenems used in the medium employed in step a) are preferably stable in a reaction medium. They are preferably chosen from: meropenem, ertapenem, doripenem, faropenem, thienamycin, biapenem, lenapenem, panipenem, razupenem, tomopenem, tebipenem, sulopenem, and the beta-methyl carbapenems described by Choi et al. in application US 2010/0160284 A1. More preferably, they are chosen from: meropenem, ertapenem, doripenem and faropenem.

Preferably, the carbapenem concentrations are between 0.05 and 32 mg/L.

More preferably, the carbapenem concentrations are between 2 and 32 mg/L for faropenem, between 0.05 and 2 mg/L for doripenem, between 0.05 and 2 mg/L for meropenem, and between 0.05 and 12 mg/L for ertapenem.

Cloxacillin corresponds to a penicillin-class antibiotic. It is used in vitro to inhibit certain beta-lactamases (Giske et al., 2010, supra). Dicloxacillin and flucloxacillin are advantageously considered as equivalent to cloxacillin. Preferably, cloxacillin is used at a concentration of between 25 and 300 mg/L.

PABN or PAbetaN corresponds to phenylalanine-arginine-beta-naphthylamide. This compound is known as an efflux pump inhibitor, making it possible, for example, to decrease the minimum inhibitory concentration (MIC) of chloramphenicol for strains of Enterobacter aerogenes (Mallea et al., 2002; Biochemical and Biophysical Research Communications 293: 1370-3). Preferably, PAbetaN is used at a concentration of between 1 and 50 mg/L.

Chromogenic substrate is to be understood to be a substrate making it possible to detect an enzymatic or metabolic activity of the target microorganisms by means of a directly or indirectly detectable signal. For direct detection, this substrate can be linked to a part acting as a fluorescent or coloured label (Orenga et al., 2009; J. Microbiol. Methods; 79(2):139-55). For indirect detection, the reaction medium according to the invention can also contain a pH indicator which is sensitive to the pH variation induced by the consumption of the substrate and which reveals the metabolism of the target microorganisms. Said pH indicator can be a chromophore or a fluorophore. As examples of chromophores, mention can be made of bromocresol purple, bromothymol blue, neutral red, aniline blue and bromocresol blue. Fluorophores include for example 4-methylumbelliferone, hydroxycoumarin derivatives or resorufin derivatives.

According to the present invention, the chromogenic substrate is preferably chosen from Indoxyl-based substrates (3-Indoxyl, 5-Bromo-3-indoxyl, 5-lodo-3-indoxyl, 4-Chloro-3-indoxyl, 5-Bromo-4-chloro-3-indoxyl, 5-Bromo-6-chloro-3-indoxyl, 6-Bromo-3-indoxyl, 6-Chloro-3-indoxyl, 6-Fluoro-3-indoxyl, 5-Bromo-4-chloro-N-methyl-3-indoxyl, N-Methyl-3-indoxyl, Aldol™, etc.); umbelliferone-based substrates (4-Methylumbelliferone, Cyclohexenoesculetin, etc.); Alizarin-based substrates; p-Naphtholbenzein-based substrates; Nitrophenol-based substrates (ortho-Nitrophenol, para-Nitrophenol, etc.); Hydroxyquinoline-based substrates; Cathecol-based substrates (Cathecol, Dihydroxyflavone, Hydroxyflavone, etc.); Resorufin-based substrates; Chlorophenol Red-based substrates; Fluorescein-based substrates; Aminophenol-based substrates (para-Aminophenol, Dichloro-aminophenol, etc.); Naphthol-based substrates (alpha-Naphthol, 2-Naphthol, Naphthol-ASBI, etc.); Aminocoumarin-based substrates (7-Amino-4-methyl-coumarin, etc.); Naphthylamide-based substrates; Acridine-based substrates (Amino-phenyl-acridine, etc.); Amino-phenoxazine-based substrates (Amino-benzophenoxazinone, Amino-pentyl-resorufin, etc.).

As an indication, the enzymatic activities targeted by the chromogenic substrates can belong to the hydrolases group, and preferably to the osidases, esterases or peptidases groups. Preferably, the enzymatic activities targeted by the chromogenic substrates are chosen from: glucuronidase, glucosidase, galactosidase, esterase, sulfatase and deaminase.

As an indication, the substrates used for the detection of a beta-glucuronidase activity can in particular be 4-Methylumbelliferyl-beta-glucuronide, 5-Bromo-4-chloro-3-indolyl-beta-glucuronide, 5-Bromo-6-chloro-3-indolyl-beta-glucuronide, 6-Chloro-3-indolyl-beta-glucuronide, Alizarin-beta-glucuronide, Cyclohexenoesculetin-beta-glucuronide or salts thereof.

The substrates used for the detection of a beta-galactosidase activity can in particular be 4-Methylumbelliferyl-beta-galactoside, 5-Bromo-4-chloro-3-indolyl-beta-galactoside, 5-Bromo-6-chloro-3-indolyl-beta-galactoside, 6-Chloro-3-indolyl-beta-galactoside, Alizarin-beta-galactoside, Cyclohexenoesculetin-beta-galactoside or their salts.

The substrates used for the detection of a beta-glucosidase activity can in particular be 4-Methylumbelliferyl-beta-glucoside, 5-Bromo-4-chloro-3-indolyl-beta-glucoside, 5-Bromo-4-chloro-3-indolyl-N-methyl-beta-glucoside, 5-Bromo-6-chloro-3-indolyl-beta-glucoside, 6-Chloro-3-indolyl-beta-glucoside, Alizarin-beta-glucoside, Cyclohexenoesculetin-beta-glucoside, Nitrophenyl-beta-glucoside, Dichloroaminophenyl glucoside or their salts.

As an indication, the substrates used to detect an esterase activity can in particular be the esters of saturated or unsaturated linear fatty acids, having between 6 and 14 carbons, preferably between 7 and 9 carbons and of 4-Methylumbelliferone, 5-Bromo-4-chloro-3-indoxyl, 5-Bromo-6-chloro-3-indoxyl, 6-Chloro-3-indoxyl, 5-Bromo-3-indolyl or of Alizarin or their salts. Preferably, they are chosen from 4-Methylumbelliferyl-octanoate, 5-Bromo-4-chloro-3-indoxyl-octanoate, 5-Bromo-6-chloro-3-indoxyl-octanoate, 6-Chloro-3-indoxyl-octanoate, 5-Bromo-3-indolyl-octanoate or Alizarin-octanoate.

The substrates used for the detection of a deaminase activity can in particular be L-Tryptophan, L-Phenylalanine, L-Tyrosine and L-Histidine.

The substrates used for the detection of a sulfatase activity can in particular be 4-Methylumbelliferyl-sulfate, 5-Bromo-4-chloro-3-indoxyl-sulfate, 5-Bromo-6-chloro-3-indoxyl-sulfate, 3-indoxyl-sulfate, phenolphthalein-disulfate or their salts.

Preferably, the chromogenic substrate is chosen from: 5-Bromo-4-chloro-3-indoxyl-beta-D-glucopyranoside (X-glucoside), 5-Bromo-6-chloro-3-indoxyl-beta-D-galactopyranoside (Magenta beta-Gal), 6-Chloro-3-indoxyl-beta-D-glucuronide (Rose beta Gur), 5-Bromo-4-chloro-3-indoxyl-N-methyl-beta-D-glucopyranoside (Green A beta Glu), Methyl-beta-D-glucopyranoside (methyl beta D glucoside) and L-Tryptophan.

Incubate is to be understood to mean raising to and holding at, for between 1 and 48 hours, preferably between 4 and 24 hours, more preferably between 16 and 24 hours, an appropriate temperature, generally of between 20 and 50° C., preferably between 30 and 40° C.

Detect is to be understood to mean discerning, with the naked eye or using an optical apparatus, the existence of a growth of the target bacteria. Advantageously, when the medium employed contains a chromogenic substrate, the detection can also make it possible to identify the target bacteria. The detection takes place using an optical apparatus for fluorescent substrates, or with the naked eye or using an optical apparatus for coloured substrates.

Specificity is to be understood to be the ability of the process or of the reaction medium to give a negative result if the target bacterial strain is not present. In other words, according to the present invention, a more specific identification corresponds to a reduction in the number of false positives linked to the strains which do not express carbapenemase, without claiming to inhibit all of these strains.

Sensitivity is understood to be the ability to give a positive result if the target bacterial strain is present in the sample.

Enzymatic resistance to carbapenems is to be understood to mean, as indicated supra, the resistance to carbapenem antibiotics due to the expression of carbapenemases by the target bacteria.

The most frequently encountered bacteria which are resistant to carbapenems are, as indicated supra: Escherichia coli, Enterobacter cloacae, Enterobacter aerogenes, Citrobacter sp., Klebsiella pneumoniae, Klebsiella oxytoca, Pseudomonas aeruginosa, Providencia rettgeri, Pseudomonas putida, Stenotrophomonas maltophilia, Acinetobacter baumanii, Comamonas sp., Aeromonas sp., Morganella morganii, Enterococcus sp., Proteus mirabilis, Salmonella senftenberg, Serratia marcescens, Salmonella typhimurium, etc.

The present invention also relates to a culture medium for detecting and/or identifying bacteria exhibiting enzymatic resistance to carbapenems, said culture medium corresponding to a basic culture medium, further comprising at least one chromogenic substrate, at least one carbapenem, cloxacillin or a combination of cloxacillin and PAbetaN.

Finally, the present invention relates to the use of cloxacillin or a combination of cloxacillin and PAbetaN for specifically detecting and/or identifying bacteria exhibiting enzymatic resistance to carbapenems.

The examples developed below aim to facilitate the understanding of the invention. They are given by way of explanation and are not intended to limit the scope of the invention.

### EXAMPLES

### Example 1

Impact of cloxacillin and/or PAbetaN on the Minimum Inhibitory Concentrations (MICs) of strains which are impermeability-resistant (IR) or exhibit another non-enzymatic resistance mechanism, or which are carbapenemase-producing, in the presence of ertapenem.

The effect of cloxacillin and PAbetaN on the MICs of the strains with ertapenem was evaluated firstly using E-test® strips in Mueller Hinton medium. A first test evaluated the impact on the MICs with ertapenem of 19 strains exhibiting non-enzymatic resistance to carbapenems (hereafter referred to as impermeability-resistant (IR)). The second test consisted in evaluating the impact of these 2 compounds on the MICs with ertapenem of 29 carbapenemases-producing strains (+1 ESBL).

1. Media and Microorganisms

Test B100904: the 19 impermeability-resistant (IR) strains belong to the following species: 10 Enterobacter aerogenes, 2 Enterobacter cloacae, 1 Citrobacter freundii, 1 Pseudomonas aeruginosa, 3 Proteus mirabilis and 2 Morganella morganii.

Test B101001: twenty-nine carbapenemase-producing strains divided as follows: 27 Klebsiella pneumoniae strains producing class A KPC carbapenemase (KPC), and 2 strains respectively of Klebsiella pneumoniae and Citrobacter freundii which produce class-B carbapenemase (NDM-1). This test also comprised an ESBL Escherichia coli strain.

The E-tests were performed in Mueller-Hinton medium supplemented or not supplemented with cloxacillin and/or PAbetaN according to Table I:

TABLE I
media tested with a view to evaluating the impact of cloxacillin and/or
PabetaN on the MICs of the IR or carbapenemase-producing strains
(90 mm Petri dish).

| | C | A | B | C |
|---|---|---|---|---|
| Mueller Hinton | 38 g/l reconstitution rate | | | |
| Cloxacillin (concentration in mg/l) | — | 200 | — | 200 |
| PAbetaN (concentration mg/l) | — | — | 25 | 25 |

2. Test
Production for each strain of an approximately 0.5 McF suspension in a 0.85% NaCl ampule.
Seeding of the 4 media by semi-automatic swabbing.
Drying of the dishes at ambient temperature for 5 to 15 min.
Application of the ertapenem E-test® strip.
Incubation for 16 to 20 hours at 37° C.

3. Results

The results are brought together in Table II for the IR strains and in Table III for the carbapenemase-producing strains.

TABLE II
impact of cloxacillin (CLX) and/or PAbetaN on the MIC of the IR strains
(comparison against MIC of the medium without cloxacillin or PAbetaN).

| | CLX | | | PAbetaN | | | CLX + PAbetaN | | |
|---|---|---|---|---|---|---|---|---|---|
| | | ≦2 MIC | >2 MIC | | ≦2 MIC | >2 MIC | | ≦2 MIC | >2 MIC |
| | No impact* | steps | steps | No impact* | steps | steps | No impact* | steps | steps |
| Enterobacter | 1 | — | 11 | 10 | — | 2 | — | — | 12 |
| Citrobacter | — | — | 1 | 1 | — | — | — | — | 1 |
| Proteus | — | — | 3 | 3 | — | — | — | — | 3 |
| Morganella | — | — | 2 | 2 | — | — | — | — | 2 |
| Pseudomonas | — | 1 | — | 1 | — | — | — | 1 | — |
| TOTAL | 1 | 1 | 17 | 17 | 0 | 2 | 0 | 1 | 18 |

*or impact which cannot be measured in the ertapenem concentration range tested (where MIC > 32)

In all cases (except 1) in which cloxacillin and/or PAbetaN has an impact of more than 2 steps on the MICs of the IR strains tested (the MIC “steps” are defined as twofold dilutions), there is a decrease in the MICs with ertapenem. In one case (E. aerogenes strain 9306074), the MIC increased in the presence of PAbetaN alone (MIC=6 without PAbetaN and MIC>32 in the presence of PAbetaN).

For the 17 strains whose MICs already decreased by more than 2 steps in the presence of cloxacillin, the effect of PAbetaN is as follows (against the MICs with cloxacillin):
7 have MICs decreased by two additional MIC steps in the presence of cloxacillin+PAbetaN
5 have less significantly decreased MICs (≦2 MIC steps)
1 is unimpacted
for 4 strains, the impact cannot be evaluated in the ertapenem concentration range tested (MIC<0.002 in the presence of cloxacillin alone, or combined with PAbetaN)

For the 2 last strains (barely impacted or unimpacted by cloxacillin alone), one is not affected by the addition of PAbetaN in addition to cloxacillin (P. aeruginosa), whilst the MIC of the other (E. cloacae) is greatly reduced in the presence of cloxacillin+PAbetaN (MIC>32 with cloxacillin alone, and MIC=0.023 with cloxacillin+PAbetaN).

TABLE III
impact of cloxacillin and/or PAbetaN on the MICs of the carbapenemase-
producing strains (comparison against MIC of the medium without cloxacillin or
PAbetaN).

| | CLX | | | | PAbetaN | | | | CLX + PAbetaN | | | |
|---|---|---|---|---|---|---|---|---|---|---|---|---|
| | | ≦2 MIC | | | | | | >2 MIC | | | | >2 MIC |
| | No | steps | >2 MIC steps | | No | ≦2 MIC steps | | steps | No | ≦2 MIC steps | | steps |
| MIC effect | impact* | negative | positive | negative | impact* | positive | negative- | positive | impact* | positive | negative | positive |
| ESBL | 1 | — | — | — | — | 1 | — | — | — | 1 | — | — |
| NDM | 2 | — | — | — | 2 | — | — | — | 2 | — | — | — |
| KPC | 7 | 16 | 2 | 1 | 3 | 14 | 2 | 7 | 2 | 17 | 3 | 4 |
| TOTAL | 10 | 16 | 2 | 1 | 5 | 15 | 2 | 7 | 4 | 18 | 3 | 4 |

*or impact which cannot be measured in the ertapenem concentration range tested (where MIC > 32)

4. Interpretation

The addition of cloxacillin into the Mueller Hinton medium makes it possible to very distinctly reduce (by more than 2 concentration steps) the MICs with ertapenem of the majority of the IR strains tested (16/19). The addition of PAbetaN alone has no impact on the majority of the IR strains tested. Conversely, the addition of PAbetaN to a medium containing cloxacillin reinforces its effect by further decreasing the MICs of the IR strains significantly (by more than 2 concentration steps for 7 strains), or more moderately (2 concentration steps or less for 5 strains).

Cloxacillin has no impact (9 strains) with ertapenem on the MICs of the carbapenemase-producing strains (NDM or KPC), or tends to reduce them slightly (16 strains). Conversely, the addition of PAbetaN alone into the Mueller Hinton medium tends to increase slightly (15 strains) or very distinctly (7 strains) the MICs of these strains with ertapenem.

Associating cloxacillin and PAbetaN makes it possible to obtain results similar to PAbetaN alone, even if the positive effect of PAbetaN on the MICs is slightly reduced by the negative effect of cloxacillin.

5. Conclusion

The use of cloxacillin alone (200 mg/L) makes it possible to significantly reduce the MICs of the majority of IR strains tested with ertapenem. The addition of PAbetaN reinforces this effect of cloxacillin.

Conversely, associating cloxacillin+PAbetaN permits an increase (moderate to strong) of the MICs of the carbapenemase-producing strains with ertapenem.

Thus, within the framework of a medium for screening for carbapenemase-producing strains, the addition of cloxacillin alone or combined with PabetaN makes it possible to better inhibit the undesirable growth of IRs, whilst having no effect, or a slightly positive effect, on the growth of the carbapenemase-producing strains.

### Example 2

Impact of cloxacillin and/or PAbetaN on the Minimum Inhibitory Concentrations (MICs) of the strains which are impermeability-resistant (IR) or exhibit another non-enzymatic resistance mechanism, or are carbapenemase-producing, in the presence of faropenem.

1. Media and Microorganisms

Seventy-seven strains of Gram-negative bacteria, of which 71 are enterobacteria and 6 non-enterobacteria (non-fermenting bacteria), whose resistance characteristics are set out in Table IV, were tested on media containing different concentrations of faropenem and cloxacillin, with or without PAbetaN, in order to establish the sensitivity and the specificity of each formulation. The tested media are described in Table V.

TABLE IV
mechanisms of resistance to antibiotics characterising the strains tested.

| | Number of strains |
|---|---|
| ESBL (7), Cephalosporinase AmpC (9) | 16 |
| Class A KPC carbapenemase (KPC) | 10 |
| Class A non-KPC carbapenemase (Class A) | 5 |
| Class B NDM-1 carbapenemase (NDM) | 15 |
| Class B non-NDM carbapenemase (Class B) | 6 |
| Class D OXA carbapenemase (OXA) | 6 |
| Impermeability resistance (IR) | 19 |

TABLE V
composition of the tested media

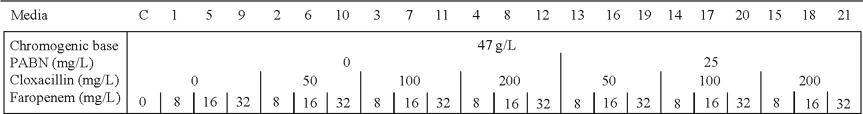

2. Test

The chromogenic media are divided into 120×120 square dishes.

The seeding is performed from 24-h pre-cultures at 37° C. on trypcase soy agar. For each strain, a 0.5 McF suspension in physiological water is produced. Each suspension is spot-seeded (1 to 2 μL) on each medium with the aid of a multi-point inoculator according to the Agar Dilution (AD) method. Readings are performed after 24 hours of incubation at 37° C.

3. Results

The results are brought together in Table VI.

An absence of bacterial growth or a number of colonies which is less than or equal to 3 are considered as corresponding to a growth inhibition. The presence of 4 colonies or more is considered to be positive growth.

TABLE VI
impact of cloxacillin and/or of PAbetaN on the growth (number of strains developing) of strains producing carbapenemases or
which are beta-lactam resistant through another resistance mechanism, in the presence of faropenem

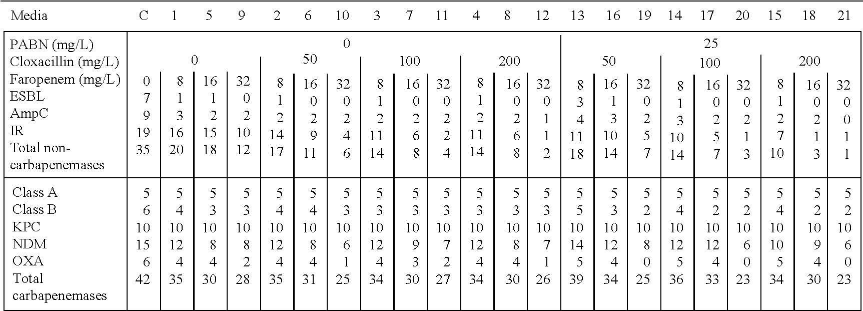

4. Interpretation

TABLE VII
sensitivity and specificity of media containing faropenem in the presence of cloxacillin, combined or not with PAbetaN,
for strains producing carbapenemases or which are beta-lactam resistant through another resistance mechanism.

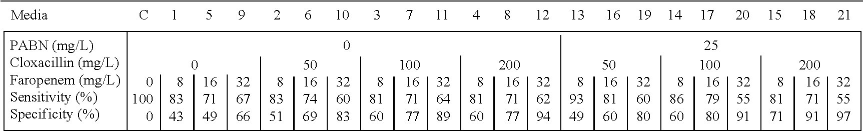

4a. Impact of Cloxacillin and PAbetaN on the IR Strains

TABLE VIII
Effects of cloxacillin, associated or not with PAbetaN, on the growth of the 19 IR strains tested.

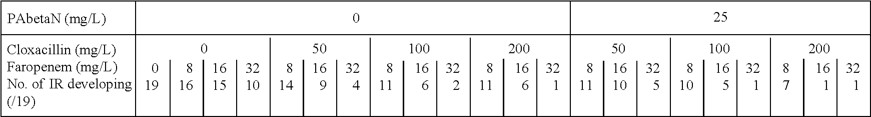

Faropenem alone has little impact on these strains, up to a concentration of 16 mg/L.

An inhibition of the IR strains is observed from the moment when 50 mg/L of cloxacillin is added, and is even more marked for the highest concentrations of faropenem tested (16 and 32 mg/L). This inhibitory effect increases with the concentration of cloxacillin (for example, for a concentration of 16 mg/L of faropenem, the addition of 50 mg/L or 200 mg/L cloxacillin respectively permits the inhibition of 6 and 9 additional IR strains). A combined effect of the concentration of faropenem and of that of cloxacillin on the IR strains is observed.

These results also confirm the better inhibition of IR strains in the presence of cloxacillin+PAbetaN. This effect is visible for the majority of the cloxacillin and faropenem concentrations tested, but is very distinctly marked in the presence of 200 mg/L of cloxacillin (whatever the faropenem concentration).

4b. Impact of Cloxacillin and PAbetaN on the Carbapenemase-Producing Strains

The results obtained in the presence of faropenem confirm that the cloxacillin alone has little or no impact on the growth of the carbapenemase-producing strains (at the 3 concentrations of faropenem tested). They also confirm that associating PAbetaN with cloxacillin makes it possible to increase the MIC of certain strains at carbapenems so long as the faropenem concentration remains less than 32 mg/L. Thus, the detection sensitivity in the presence of 8 or 16 mg/L of faropenem associated with 50 or 100 mg/L of cloxacillin is greater in the presence of PAbetaN. For 200 mg/L of cloxacillin and 8 or 16 mg/L of faropenem, the addition of PAbetaN does not change the detection sensitivity.

5. Conclusion

These results confirm the inhibitory role of cloxacillin on the strains which are impermeability resistant, in the presence of a carbapenem. These results also confirm the superior inhibition of the IR strains when cloxacillin is associated with PAbetaN. The addition of cloxacillin and PAbetaN thus makes it possible to very greatly increase the specificity of the medium.

The addition of cloxacillin (at the 3 concentrations tested) and of PAbetaN (25 mg/l) into the medium does not alter the detection of the carbapenemase-producing strains in the presence of 8 or 16 mg/L faropenem. It is confirmed that the addition of PAbetaN tends to improve their detection at faropenem concentrations equal to 8 or 16 mg/L, associated with 50 or 100 mg/L cloxacillin.

In the presence of faropenem, the results observed with ertapenem (E-test) are therefore confirmed, namely better inhibition of the IR strains by addition of cloxacillin and an improvement of the detection of the carbapenemase-producing strains in the presence of PAbetaN.

1. A process for detecting carbapenemase-producing bacteria in a biological sample comprising:
contacting said sample with a reaction medium comprising at least one carbapenem and cloxacillin,
incubating the whole so as to allow carbapenemase-producing bacteria to grow, and
detecting the strains corresponding to carbapenemase-producing bacteria.

2. A process for detecting and/or identifying carbapenemase-producing bacteria in a biological sample comprising:
contacting said sample with a reaction medium comprising at least one carbapenem, cloxacillin, and at least one chromogenic substrate,
incubating the whole so as to allow carbapenemase-producing bacteria to grow, and
detecting the strains corresponding to carbapenemase-producing bacteria.

3. The process according to claim 1, wherein the reaction medium also comprises phenylalanine-arginine beta-naphthylamide (PAbetaN).

4. The process according to claim 1, wherein the carbapenems are chosen from: ertapenem, meropenem, doripenem and faropenem.

5. The process according to claim 1, wherein the cloxacillin concentration is between 25 and 300 mg/L.

6. The process according to claim 3, wherein the PAbetaN concentration is between 1 and 50 mg/L.

7. The process according to claim 2, wherein the chromogenic substrate detects an activity chosen from: glucuronidase, glucosidase, galactosidase, esterase, sulfatase and deaminase.

8. The process according to claim 2, wherein the chromogenic substrate is chosen from: 5-Bromo-4-chloro-3-indoxyl-beta-D-glucopyranoside (X-glucoside), 5-Bromo-6-chloro-3-indoxyl-beta-D-galactopyranoside (Magenta beta-Gal), 6-Chloro-3-indoxyl-beta-D-glucuronide (Rose beta Gur), 5-Bromo-4-chloro-3-indoxyl-N-methyl-beta-D-glucopyranoside (Green A beta Glu) and L-Tryptophan.

9. The process according to claim 1, wherein the reaction medium is a liquid or solid culture medium.

10. A culture medium for detecting and/or identifying bacteria exhibiting enzymatic resistance to carbapenems, comprising a basic culture medium, at least one chromogenic substrate, at least one carbapenem and cloxacillin.

11. The culture medium according to claim 10, further comprising phenylalanine-arginine beta-naphthylamide (PAbetaN).

12. (canceled)
